(19) United States
(12) Patent Application Publication
(10) Pub. No.: US 2025/0229021 A1
(43) Pub. Date: Jul. 17, 2025

(54) Intuitive Total Intravenous Anesthesia Infusion System and Method of Operation

(71) Applicant: Craig Anthony Lampe, Lubbock, TX (US)

(72) Inventor: Craig Anthony Lampe, Lubbock, TX (US)

(21) Appl. No.: 19/025,338

(22) Filed: Jan. 16, 2025

Related U.S. Application Data
(60) Provisional application No. 63/621,520, filed on Jan. 16, 2024.

(51) Int. Cl. A61M 5/172; A61M 5/14; G16H 10/60; G16H 20/17

(52) U.S. Cl. CPC A61M 5/172; A61M 5/1413; G16H 10/60; G16H 20/17; A61M 2005/1402; A61M 2205/123; A61M 2205/18; A61M 2205/502

(57) ABSTRACT

Methods, systems, and apparatus, including computer programs encoded on computer storage media related to an intuitive total intravenous anesthesia infusion system and method of operation. The system includes a device that has a body with a display device, a rotatable input control knob, a cartridge receiver, and one or more processors. The one or more processors are configured to set dosing parameters for a medication and dispense the medication via a medication dispensing controller according to the dosing parameters.

### CROSS-REFERENCE TO RELATED APPLICATIONS

This application is a non-provisional application and claims the benefit of provisional U.S. Patent Application No. 63/621,520, filed Jan. 16, 2024, which is hereby incorporated by reference in its entirety.

### FIELD

This application generally relates to an anesthesia infusion system, and more specifically relates to an intuitive total intravenous anesthesia infusion system and method of operation.

### BACKGROUND

Inhalation anesthetic agents (such as desflurane, sevoflurane, and isoflurane) used for sedation have an unfavorable post-operative nausea and vomiting effect (PONV). This PONV has a negative impact on patients and a large negative economic effect on healthcare delivery, causing patient suffering and requiring patients to stay in healthcare systems longer than required to receive many, sometimes expensive, medications to mitigate this PONV.

However, using inhalational anesthesia agents is currently favored by users due to the simplicity and effectiveness of use. A vaporizer adds these halogenated gases to a fresh gas flowing to a patient. Current devices are easy to use and include a large knob on top of the device, allowing a single hand motion to begin, adjust, and end delivery of medication. Similarly, nitrous oxide (N₂O) can be delivered with the turn of a single knob. Despite inhalation anesthesia agents being a suboptimal choice of medication, some of the popularity and prevalence of using these medications is due to ease of use. Furthermore, these medications are readily available for abuse as current anesthesia machines lack a lockout mechanism. Leftover inhalation agents are easily accessible as the vaporizer is difficult to empty and seldom removed from the machine. Furthermore, inhalational agents and N₂O are exceptionally environmentally hazardous—desflurane creates more than 2550 times more greenhouse gas effect than carbon dioxide (CO₂) emissions. Reduction in use of these inhalational agents is an ethical environmental imperative. As an alternative, intravenous (IV) agents can be used for sedation, including general (total) anesthetics. This is referred to as total intravenous anesthesia (TIVA). Medications utilized for TIVA include propofol, etomidate, and dexmeditomidine. Propofol is the most frequently used IV agent due to its fast mechanism of action, favorable PONV profile, and reasonable cost.

IV sedation medications have many beneficial characteristics: they tend to provoke less PONV (especially propofol) and their use has negligible environmental impact, especially when compared to inhalational anesthetic agents and N₂O. Currently, generic IV pumps are utilized to run these IV sedation medications, when needed. This is a cumbersome, time-intensive process wherein the IV pump must be extensively programmed to start TIVA. Current generic pumps are not purpose-built for this task. The medicine must be manually flushed through the tubing (i.e., referred to as priming), which is a frustrating, time-intensive process. Frequently, priming does not remove all air bubbles from the fluid line, which is dangerous to the patient. If the pump is used to attempt to flush tubing, the included air detector inevitably and uselessly alarms multiple times even though no patient is attached. Some pumps require medicine to be drawn into syringes for infusion. These must be refilled and the pump reprogrammed on a frequent and distracting basis throughout the sedation. These syringes frequently mechanically jam as medication makes plunger materials swell and syringe motion tighten. At the end of the sedation, the IV sedation agents must be removed and disposed of manually, which is a time-intensive process. To focus on patient safety, anesthesiologists should spend their time planning and executing the anesthetic, not dealing with frustrating and inefficient setup and disposal steps.

In view of the foregoing, it is apparent that a need exists in the art for an intuitive total intravenous anesthesia infusion system and method which overcomes, mitigates or solves the above problems in the art. It is a purpose of this invention to fulfill this and other needs in the art which will become more apparent to the skilled artisan once given the following disclosure.

### BRIEF SUMMARY OF THE INVENTION

In general, one innovative aspect of the subject described in this specification can be embodied in systems, computer readable media, and methods for an intuitive total intravenous infusion system and method of operation. The system includes a device comprising a body with a display device, a rotatable input control knob, a cartridge receiver, and one or more processors. The one or more processors are configured to recognize and then set dosing parameters for a medication and dispense the medication via a medication dispensing controller according to the dosing parameters.

In some aspects of the system, the device controls the operation of the cartridge to dispense medication using the medication dispensing controller in the form of a pump housed in the cartridge. The cartridge is seated into a cartridge receiver that includes an interface that mates to the cartridge and controls the operation of the pump.

In some configurations, the cartridge may include a reservoir that is preloaded with an amount of sterile medication. In other configurations, the cartridge does not include any preloaded medication, but the cartridge receives medication via one or more sterile input ports. For example, the cartridge may receive medication via a medication bottle and/or a syringe with Luer Lock adapter. In any of the configurations, the pump causes the medication to be dispensed via the cartridge through output ports to tubing for administration of the medication to a patient.

The appended claims may serve as a summary of this application.

Further areas of applicability of the present disclosure will become apparent from the detailed description, the claims and the drawings. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIGS. 1A-1B illustrates an exemplary environment where embodiments of an intuitive total intravenous anesthesia infusion system may operate.

FIG. 2A illustrates a top perspective view of an exemplary intuitive total intravenous anesthesia infusion system.

FIG. 2B illustrates a side view of an exemplary intuitive total intravenous anesthesia infusion system.

FIG. 2C illustrates a side view of an exemplary intuitive total intravenous anesthesia infusion system.

FIG. 3A illustrates an exemplary cartridge that may be used according to some embodiments.

FIG. 3B illustrates a block diagram of an exemplary cartridge that may be used according to some embodiments.

FIGS. 4A-4C illustrates an exemplary cartridge that may be used according to some embodiments.

FIG. 5 illustrates a block diagram of an exemplary cartridge that may be used according to some embodiments.

FIG. 6 illustrates a block diagram of an exemplary cartridge that may be used according to some embodiments.

FIG. 7 illustrates a block diagram of an exemplary cartridge that may be used according to some embodiments.

FIG. 8 illustrates a block diagram of an exemplary cartridge that may be used according to some embodiments.

FIG. 9 illustrates perspective views of exemplary spikes that may be used according to some embodiments.

FIG. 10 illustrates an exemplary method of operation of an intuitive total intravenous anesthesia infusion system.

FIG. 11 illustrates an exemplary method of operation of an intuitive total intravenous anesthesia infusion system.

FIG. 12 illustrates an exemplary computer system for performing any one or more of the methodologies discussed herein.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

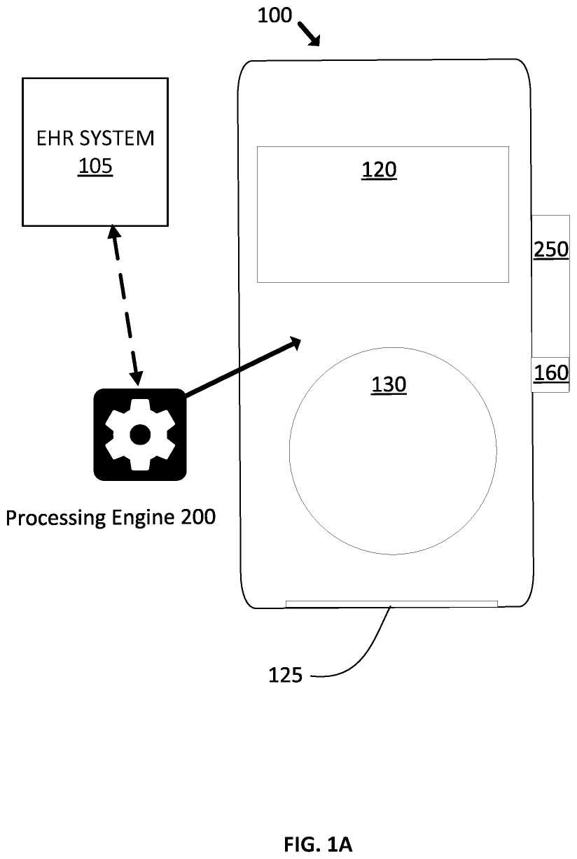

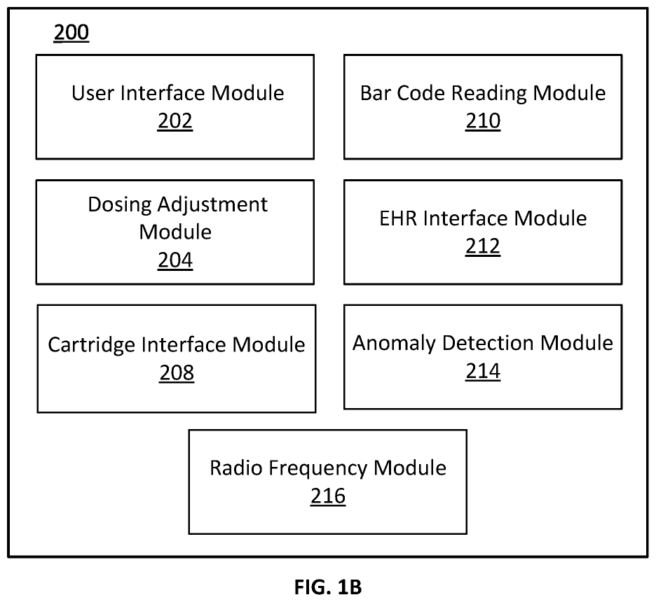

Referring to FIGS. 1A-1B, the diagrams illustrate an exemplary environment in which some embodiments of an intuitive total intravenous anesthesia infusion system may operate.

The exemplary device 100 comprises a touch screen display device 120, an input control in the form of a rotatable knob 130, one or more onboard processors, a cartridge receiver 160 and a removable cartridge 250 that may be placed into and removed from the cartridge receiver 160. In some embodiments, the device 100 includes a barcode reader 125 which may be positioned at a bottom portion and/or a top portion of the device. In some embodiments, the device has a barcode reader on both the top and bottom portion of the device.

The device 100 includes a processing engine 200 that may be performed by the one or more onboard processors. The processing engine 200 is configured to perform control and operation of the device 100 and controls the dispensing of medication via the removable cartridge 250. In some embodiments, processing engine 200 may perform the methods 400, 500 or other methods described herein. In some embodiments, the device 100 is a computing device capable of hosting and executing one or more applications or other programs capable of sending and/or receiving information. The processing engine 200 comprises multiple software modules that may execute some of the functionality described herein.

The User Interface Module 202 provides system functionality for presenting a user interface via the touch screen display device 120. User inputs received by the user interface herein may include clicks, keyboard inputs, touch inputs, taps, swipes, gestures, voice commands, activation of interface controls, Bluetooth or Wi-Fi control from a separate smart device such as a mobile phone, ring, watch, or tablet, and other user inputs. Outputs may go through a built-in speaker, through a Bluetooth connection to headphones or other smart mobile device. In some embodiments, the User Interface Module 202 presents a visual user interface on a display screen of the display device 120.

The Dosing Adjustment Module 204 provides system functionality for controlling and managing dosing parameters for dosing of a medication via the device 100. For example, the dosing parameters may include an amount (such as volume or mass) of a medication, a weight of a patient to whom the medication is to be administered, a total amount of the medication to be administered, a flow rate of the medication over time to be administered, time intervals to provide a periodic dose of an amount of a medication, and/or an amount of a bolus dose of the medication.

The Cartridge Interface Module 208 provides system functionality for interfacing and communication with an inserted cartridge 250 in the cartridge receiver 160. This module controls the operation of the cartridge 250 according to the dosing parameters set by a user of the device 100.

The Bar Code Reading Module 210 provides system functionality for reading information from bar codes and storing the information on the device 100 and/or for communication of the information to a separate electronic health record (EHR) system. This module may include a button, switch, ultrasonic, or motion sensor for activating the laser for reading bar codes.

The Radio Frequency Module 216 provides system functionality for receiving and/or reading data using near field communication subsystem. For example, the device may read near-field communication (NFC) tags, or other passive devices that emit a signal (such a user identification badge).

The EHR Interface Module 212 provides system functionality for securely communicating information (in an encrypted HIPPA-compliant manner) between the separate EHR system and the device 100.

The Anomaly Detection Module 214 provides system functionality for continuously monitoring for anomalous situations. For example, the device 100 may monitor for: air detected in a fluid pathway of the cartridge 250; the amount of fluid received from an attached medication bottle to the cartridge and whether a bottle is empty and/or near empty; a low battery voltage of the device; and/or detecting pressure indicating an occlusion situation on the patient side of a dispensed medication.

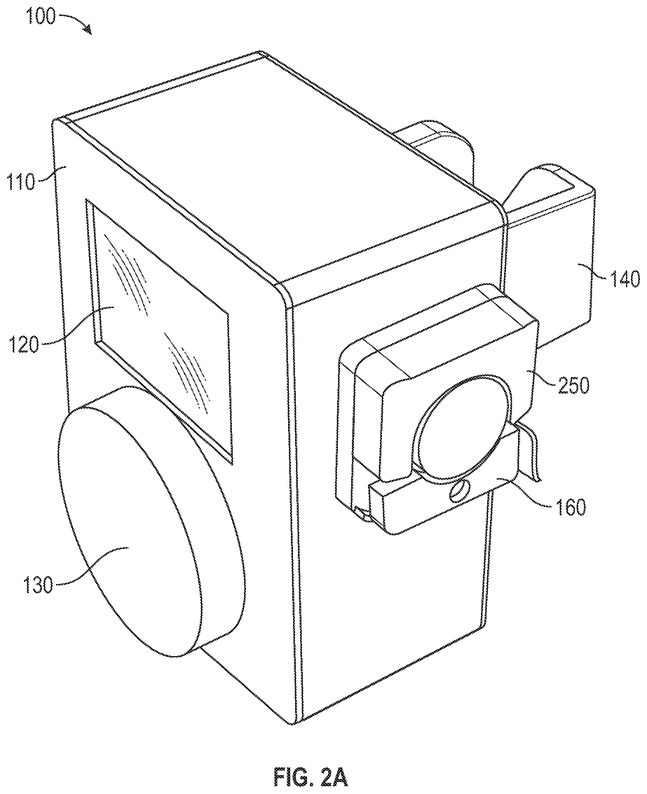

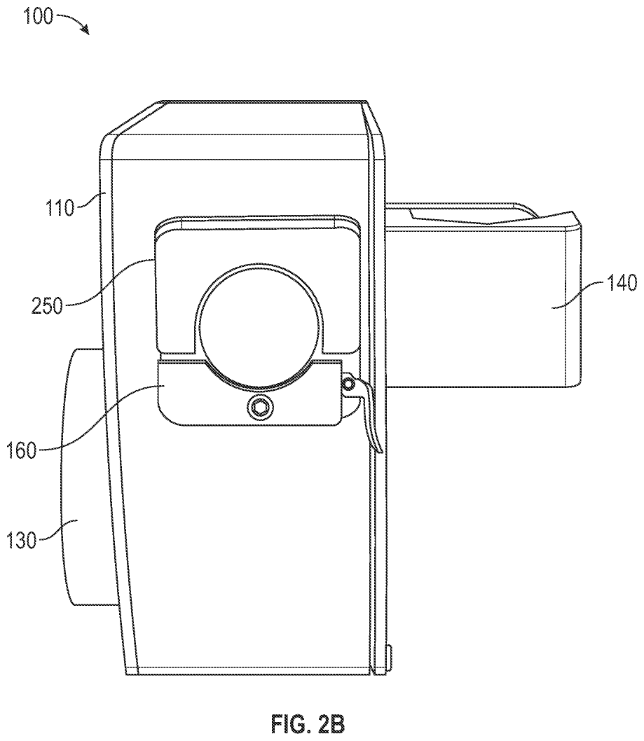

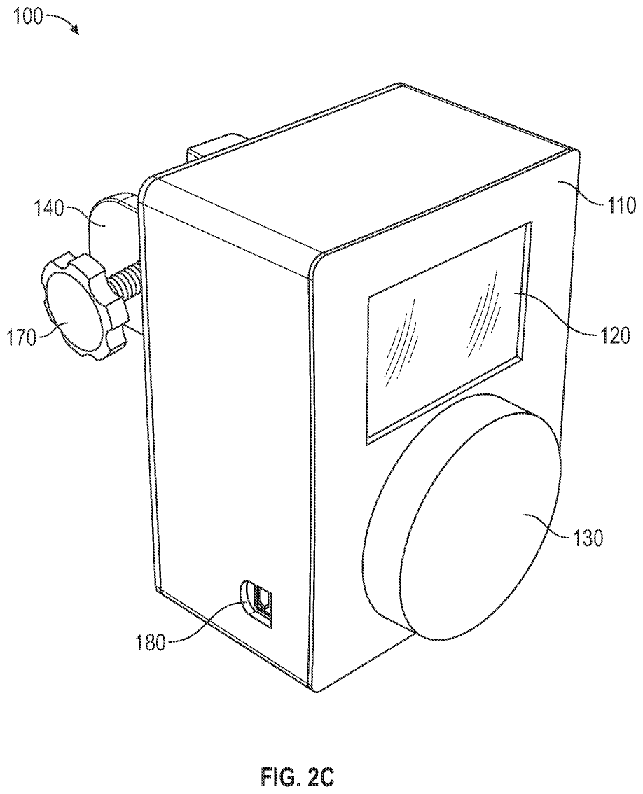

Referring to FIGS. 2A-2C, according to an exemplary embodiment, the device 100 comprises a touch screen display device 120, an input control in the form of a rotatable knob 130, an onboard processor and battery, a cartridge receiver 160 and one or more disposable cartridges 250 that may be placed into and removed from the cartridge receiver 160. In some embodiments, the device has a clamping or attachment mechanism 140 to attach the device 100 to a pole, infusion stand or drip stand. For example, the attachment mechanism 140 may have a screw knob 170 or other clamping system to secure the attachment mechanism 140 to a stand.

In some embodiments, the device 100 includes circuitry such as wireless subsystem, an RS 232 serial port 180, an internal battery, a real time clock, storage device (e.g., a secure digital (SD) card), a radio frequency identification (RFID) reader or an NFC reader and optional biometric identification components. The wireless subsystem allows the device 100 to securely and wirelessly communicate with an EHR system. This allows minute-by-minute medication dosing to be documented from the device 100 to the HER, and this allows parameters (e.g., patient weight) to be transferred from the EHR to the device interface for use in dosing calculations. Moreover, the wireless subsystem provides for integration and communication with a mobile device and a mobile application (mobile app) on the device. For example, the mobile app may be configured to and control the operation of the device 100 (such as changing patient weight, current dose, requesting a bolus delivery, silencing alarms, and other settings through the mobile app). Some of the wireless data communications to and from the device 100 (those requiring HIPPA protection) are encrypted in a manner to comply with HIPPA regulations. Bluetooth function may also be used to attach headphones so that audible alarms are heard only by the anesthesia professional or other preselected health professional, keeping distracting noises minimal for other operating room or intensive care unit (ICU) staff. The RS 232 serial port allows for wired data communication to the EHR system (other ports may be used, such as USB, etc.). The internal battery allows for backup power to the device 100. The device 100 may operate directly from the internal battery or from a wired connection to an external power source. The internal battery charges when the device 100 is connected to the external power source. The real time clock is set automatically through Wi-Fi functionality and is used by the device to provide accurate date and time stamps for recording data and information generated by the device 100. The device 100 may include onboard permanent and/or removable storage. The storage is used by the device to save operational data of the device 100 and configuration settings for different users of the device. Onboard storage may also be used to cache/store data to and from EHR interface in case of momentary losses of wireless or wired connection. This data will be sent and confirmed upon resumption of data connection.

In some embodiments, the device 100 may have one or more external battery packs which can be quickly replaced to extend functionality with or without access to wall power.

In some embodiments, the device 100 includes subsystems and functionality for biometric identification of a user. For example, the device 100 may use biometrics, such as fingerprints, facial recognition, retina scans, etc. as a user identification method, to appropriately identify the user, then store and/or load user preferences. These user preferences and their associated biometric identification methods may be uploaded to a cloud system to be shared among multiple device 100 units in a variety of locations. For example, this would allow devices 100 to remain in sedation locations, rather than requiring devices 100 to be moved with anesthesia professionals as they change locations.

In some embodiments, the device 100 includes a barcode reader 125 positioned at the bottom or other location of device 100. In some modes of operation, a user may scan, via the barcode reader 125, barcodes for medication bottles and/or staff or patient identifiers. This method may be used to expedite linking to an appropriate EHR workstation in the same location. In other embodiments, a separate wired or wireless barcode reader may be connected to device 100 to read/scan barcodes (for example by USB interface).

Additionally, the device 100 may obtain medication information and/or patient information/parameters directly from an integrated EHR system. This interface may allow warnings to pass from EHR to the device 100 (for example, “medication not due yet,” forcing a pause, stop, or override through input interface if needed)

In some embodiments, the touch screen display device 120 displays information related to the operation of the device 100. For example, the display device 120 receives user input for pausing, resuming, or changing the rate of infusion of medication. The display device 120 receives user input for changing settings/options. The display device 120 clearly displays current dosing and information related to changes to dosing of the medication, including “high” and “low” dosage warnings. The display device 120 displays other information related to total medication infused, time until bottle is empty, and performance of and/or faults detected by the device 100. In some embodiments, display device 120 may also show various parameters which assist the anesthesia professional in providing care, for example, total dose of propofol administered and time to full recovery (TTFR), which is an estimate of time to wake-up time.

In some embodiments, the input control 130 is a rotatable knob with or without a push function for selecting items displayed via the display device 120. Changes to values may be made via the input control 130 to dose, weight, other values and other options. Depending on the mode of operation of the device 100, changes occur via input from a user (i.e., an anesthesiology professional) manually spinning or turning the knob 130, and pause/resume/confirm functions may occur via the user manually pushing the knob. The input control 130 and display device 120 are used together to make selections and provide inputs to the device 100 for the dosing and dispensing of medication via a cartridge 250. In some embodiments, timers may be set to stop the medication and/or alert the user after a predetermined time. In some embodiments, a volume to be infused (VTBI) can be set and the pump will stop the infusion and/or alert the user when that total amount has been administered to the patient (e.g., a predetermined amount of intravenous fluids (IVF) to be given to the patient). In some embodiments, the pump software will be able to calculate and then infuse a fluid bolus calculated to be equal to the nil per os (NPO) (i.e., nothing by mouth) fluid deficit (e.g., maintenance IVF rate multiplied by the number of hours the patient has been NPO, which may be adjusted using patient nomograms).

In some embodiments, a cartridge receiver 160 is formed integrally with or attached to a body 110 of the device 100. The cartridge receiver 160 includes a reusable motor that interfaces with the cartridge 250. In some embodiments, the reusable motor includes a mechanical interface such as a gear attached to the motor. This interface interacts mechanically with the cartridge which controls the operation of the pump 254. In some embodiments, a motor is constructed inside and included in the disposable cartridge 250 itself, having an electronic interface with the cartridge receiver 160.

In some embodiments, the cartridge 250 can have both a reusable and a disposable portion: 1) the disposable portion consists of a new sterile fluid pathway, which can be replaced a number of times to reduce total cost and environmental impact; and 2) the reusable portion consists of all other components of the cartridge being reusable.

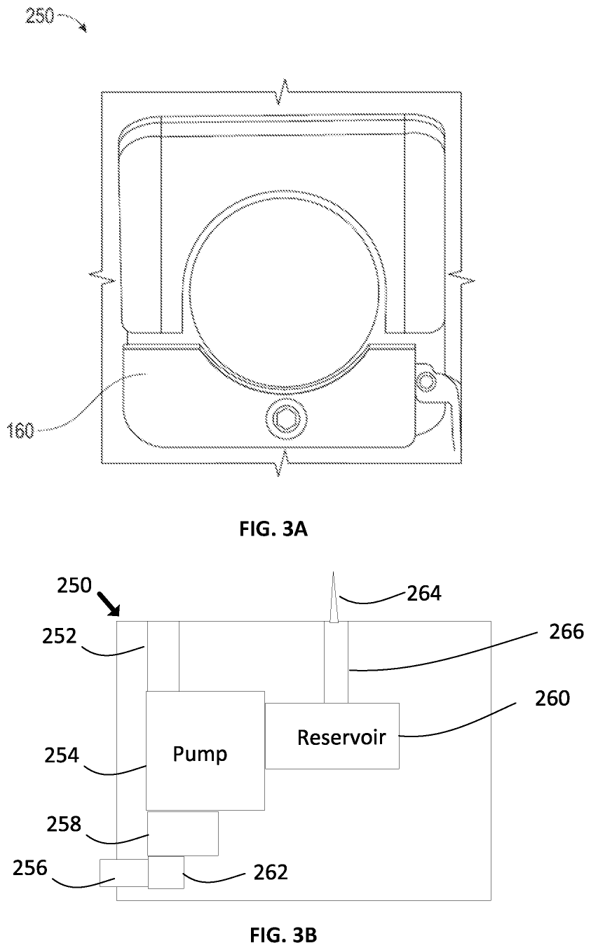

Referring to FIGS. 3A-3B, according to an embodiment, an example removable cartridge is illustrated. In some embodiments, the device 100 uses disposable cartridges 250 to dispense medication via a medication dispensing controller 254. In some embodiments, the cartridge 250 is universal and includes a chamber or reservoir 260 to receive a fluidic medication solution. The cartridge 250 includes tubing enclosed within a body of the cartridge where the tubing is used to move medication from one or more input ports to a patient output port. In some embodiments, a peristaltic pumping mechanism is utilized.

In some embodiments, the cartridge 250 may include one or more rigid spikes 264 at a top surface of the cartridge to attach a bottle of fluidic medication. For example, a bottle with a rubberized stopper (facing downward) may be placed onto the spike 264 of a cartridge. The spike penetrates the rubberized portion of the stopper thereby allowing the fluidic meditation to flow into the cartridge reservoir 260 via a fluid channel 266 and simultaneously allowing air to flow back into the bottle as needed to prevent a vacuum. Air can be detected and rejected within the cartridge 250 by an air detector 258. In some embodiments, the cartridge may include multiple spikes 264 set at different heights to allow only one bottle of medication to empty at a time, which preserves the ability to infuse medication thereby assisting the anesthesia professional.

In some embodiments, the cartridge 250 includes a medication dispensing controller 254 (e.g. internal pump mechanism) that is actuated via an interface between a connector (such as an electric or mechanical connection) between the cartridge receiver 160 and the cartridge 250. The device 100 controls the operation of the medication dispensing controller 254 and provides management of fluid administration of the medication.

In some embodiments, the device 100 determines or detects the removal and/or replacement of a new medication bottle onto the cartridge spikes. In some embodiments, the reservoir 260 of a cartridge 250 is pre-loaded with an amount of medication. A cartridge may be pre-loaded with various amounts of medication (e.g., 100 mL, 200 mL, or 500 mL of propofol; or 100 mL of IV acetaminophen). One or more bottle spikes may be used for replacement of medication as additional medication is needed for longer sedation. In some embodiments, both a prefilled reservoir and one or more bottle spikes are present, allowing medication to be quickly started (from the included reservoir), and allowing additional medication to be added (utilizing one or more bottle spikes) as sedation and infusion time extends.

In some embodiments, the cartridge 250 includes an identifier. For example, the device 100 may read or obtain the cartridge identifier. The cartridge identifier may include one or more data including: exact medication and concentration, a unique cartridge number, a date the cartridge (or disposable portion) was made, a date when a medication was placed into the reservoir of the cartridge, a date and time when medications would expire (allowing an automated safety system to reject a cartridge and alarm if medication is expired), a description of the medication placed into the reservoir, and/or other attributes of the medication. The device 100 obtains the cartridge identifier and associated cartridge information. In some embodiments, the device 100 performs a configuration or initialization operation of the device 100 based on the information read from the cartridge 250. This initialization action will pump medication into the tubing, removing air and preparing the tubing for safe and immediate patient use, saving time, effort and assisting the anesthesia professional. In some embodiments, the cartridge 250 includes an output port 256 fluidly connected to the reservoir 260 where an amount of the medication is pumped through the output port. Flexible plastic tubing may be connected to the output port 256. In some embodiments, low dead space (low volume) tubing which connects to a patient may be pre-attached to the cartridge 250, allowing an initialization/priming/de-air maneuver.

In some embodiments, the cartridge 250 may be configured with an input port 252 that has a needle port or adapter (e.g., an industry standard Luer Lock adapter) to allow the introduction of additional medication, such as through a syringe. For example, a needle port or Luer Lock adapter allows for sterile addition of medication which then quickly makes its way to the patient connection. This allows an anesthesia provider to administer additional medication quickly and easily at the pump mechanism. In some embodiments, there is an injection detector on or near the needle port or Luer Lock adapter to detect that a medication was added (e.g., triggering a wireless or wired signal to the EHR interface which may trigger a dialog to document medication and dosage). In some embodiments, the patient pressure sensor is used to detect introduction or injection of additional medications at this injection adapter.

In some embodiments, the cartridge 250 includes an air detector 258 inside the cartridge to detect unintentional air in the fluid path to the output port 256. In some embodiments, the pump may be reversed to remove air from the fluid delivery path of the medication, thereby diverting the air away from a patient. In some embodiments, a tubing path inside the cartridge 250 is configured to connect to a spike, which connects to medication bottles, to allow for clearance of any detected air without negatively impacting sterility. In some embodiments, detection of air may trigger a pause and/or an audible or haptic alarm on the device 100 or other connected device (EHR or smart device). In some embodiments, when air is detected by the air detector 722, a patient solenoid 730 is closed and an air elimination solenoid 728 is opened, allowing the pump 750 to divert detected air back to the air eliminator 504 and avoid air bubbles traveling to the patient. Once air is cleared, a patient solenoid 730 is opened and an air elimination solenoid 728 is closed to resume normal pumping operation to the patient.

In some embodiments, the cartridge 250 includes a safety pressure sensor 262 coupled to the output port 256 (i.e., the patient side of the tubing) to detect occlusion of flow. This helps to prevent and/or detect intravenous (IV) catheter infiltration. In some embodiments, the device 100 continuously monitors flow rate and/or pressure of the fluid being dispensed from the device 100. The device 100 may provide audible safety alerts via a speaker (not illustrated) to inform an operator of the device's reduced or stopped flow rate and/or anomalous pressure situation. These audible alerts may further be transmitted to the EHR for alert and/or documentation, a remote monitoring location, and/or a smart device or headphones if connected.

In some embodiments, the device 100 comprises multiple cartridge receivers to control multiple cartridges (e.g., to simultaneously run IVF and/or to infuse single doses of additional medications needed during sedation, such as an adult unit dose of nausea medication). Alternatively, a cartridge 250 may include a separate fluid pathway to also provide the IVF. For example, the device 100 would allow IVF (which is the “carrier fluid”) to be given automatically at patient “maintenance level” (for example, “maintenance IVF”). This is equal to the “4:2:1 calculation” (i.e., weight based; 40 mL/hr for the first 10 kg, 20 mL/hr for the next 10 kg, 1 mL/kg/hr for the remainder of the patient's weight). The device 100 would also be able to briefly infuse boluses of IVF if activated to further guarantee delivery of doses of drug (propofol) without any delay. Other/secondary cartridge receivers and appropriate cartridges could be used to infuse other necessary medications without removing or interrupting the primary infusion of sedation medication.

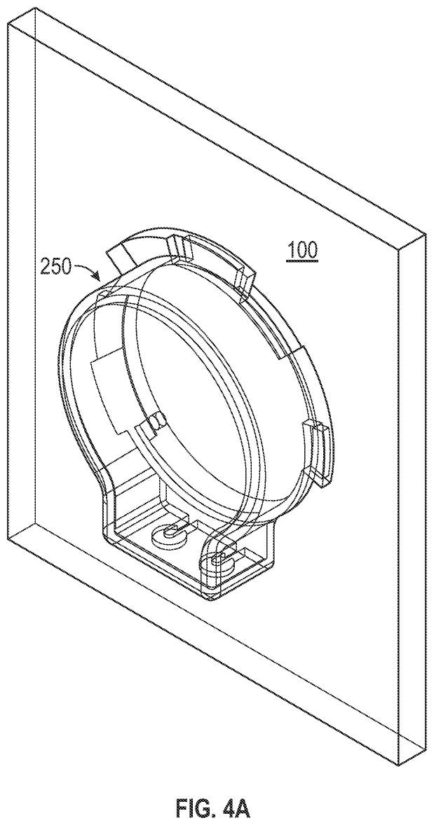

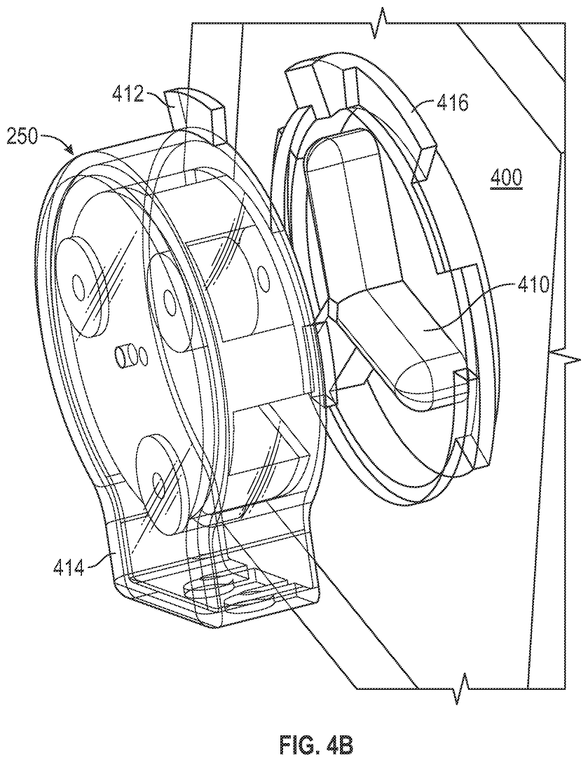

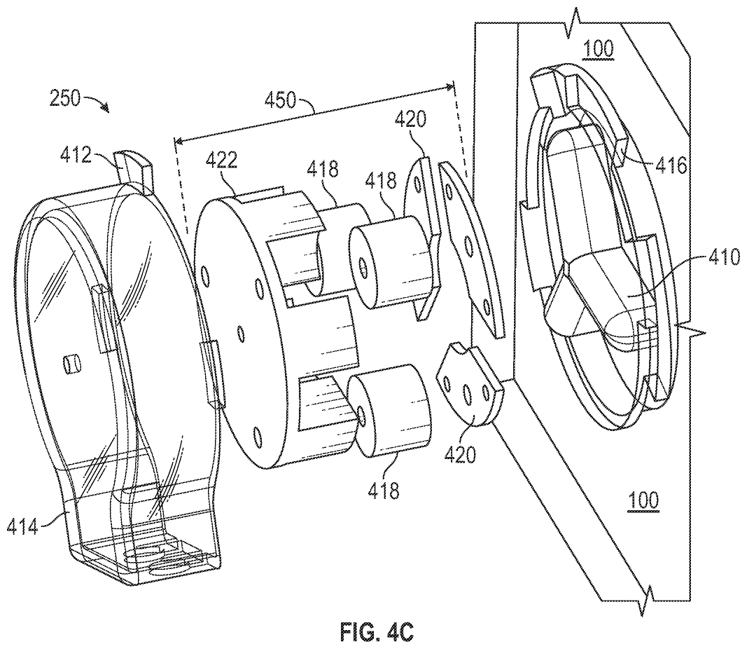

FIGS. 4A-4C illustrate an exemplary cartridge that may be used according to some embodiments. In some embodiments, the cartridge 250 includes a cartridge housing 414 with multiple flanges 412 that seat into a portion of the device body 400. The device body 400 has a matching number of receiving areas 416 to receive the multiple flanges 412. The flanges 412 are seated into the receiving areas and the cartridge body 250 is rotated to friction lock the cartridge into the device body 400. In some embodiments, the cartridge body includes a spring-loaded locking post that when pushed inwards towards the device body 400 allows rotation of the cartridge 250 for removal. Otherwise, the locking post prevents rotation of the cartridge 250. In some embodiments, a lock and key mechanism is necessary to remove the cartridge 250 from the device body 400 (for example to secure abusable medications).

In a depicted embodiment, a rotatable gear 410 having a number of gear arms fits within a portion of the cartridge when the cartridge 250 is seated into the device body. The device 100 controls the rotation of the gear 410 to control the pump 450.

In some embodiments, the pump 450 is in a form of a peristatic pump (e.g., a roller pump) with rollers 418. Flexible tubing is disposed through a portion of the pump to dispense medication. The rollers 418 are placed between a rolling frame 422 and rolling frame end caps 420. The gear 410 rotates the rolling frame 422 to cause the rollers 418 to rotate where the rollers 418 provide a compressive force against the flexible tubing. This causes fluid in the flexible tubing to move through the tubing and other tubing connected to the cartridge 250. The cartridge 250 includes tubing disposed within a body of the cartridge where the tubing is used to move medication from one or more input ports to a patient output port and/or a disposal port.

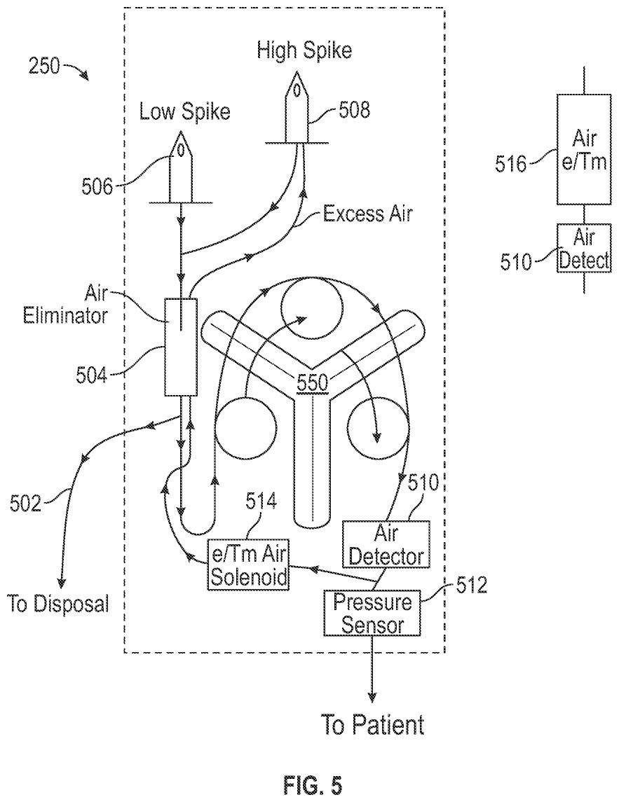

FIG. 5 illustrates a block diagram of an exemplary cartridge that may be used according to some embodiments. This example is a modification of the cartridge depicted in FIGS. 4A-4B.

In some embodiments, the cartridge 250 includes a low spike 506 port and a high spike 508 port. The cartridge includes an air eliminator 540 configured to remove air from tubing connected via the air eliminator 540.

In some embodiments, the cartridge 250 includes an air detector 510, a pressure sensor 512 and an eliminator air solenoid. As shown, in the offset figure an air eliminator 516 may be placed in a position in between the pump 550 and the air detector 510. The air detector detects air in the fluid pathway. The pressure sensor determines a pressure value of the fluid in the fluid pathway. In some embodiments, the air detector determines a refractive index of fluid in order to determine the occurrence of a medication or air in the fluid pathway.

In some embodiments, the cartridge 250 contains a separate fluid path designed for medication disposal. This interfaces with a separate fluid path 502 on the device 100 which contains tubing and a mechanical system to pump fluid. This fluid path 502 goes into a permanent disposal container (not depicted), which may be comprised of absorptive, poisonous, and caustic chemicals to prevent theft/diversion of medication for illegal use/abuse (e.g., at the conclusion of use with a patient, this pathway disposes all remaining and/or unused medication into a disposal bin, where the medication is no longer available for theft/diversion/abuse).

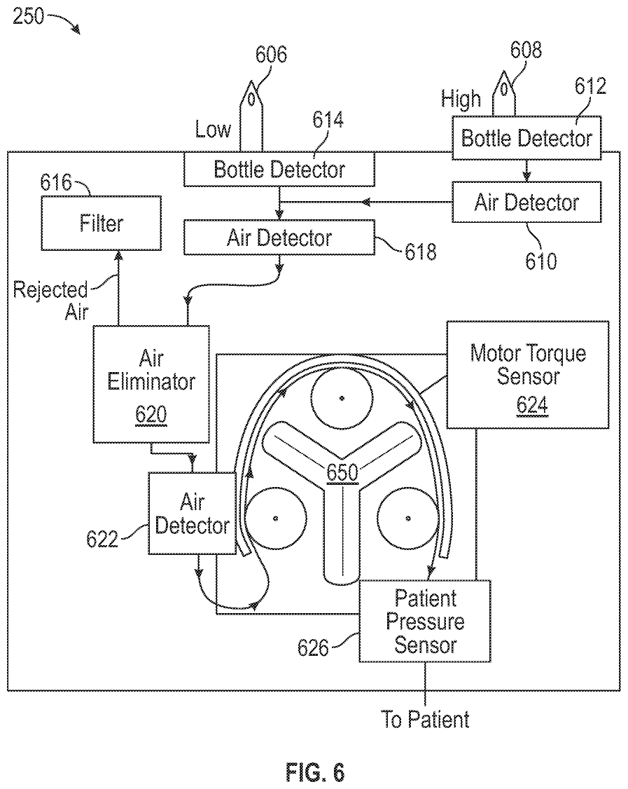

FIG. 6 illustrates a block diagram of an exemplary cartridge that may be used according to some embodiments. This example is a modification of the cartridge depicted in FIGS. 4A-4B.

In some embodiments, the cartridge 250 includes a low spike 606 port and a high spike 608 port. The cartridge includes air detectors 610 and 618, bottle detectors 612 and 614, a filter 616, an air eliminator 620, a motor torque sensor 624, and a patient pressure sensor 626. The air detectors 610 and 618 detect air in the fluid pathway. The bottle detectors 612 and 614 detect the presence of a medication bottle attached to the spikes 606 and 608. A filter 616 filters discharge of air (with possible fluid) from the cartridge 250.

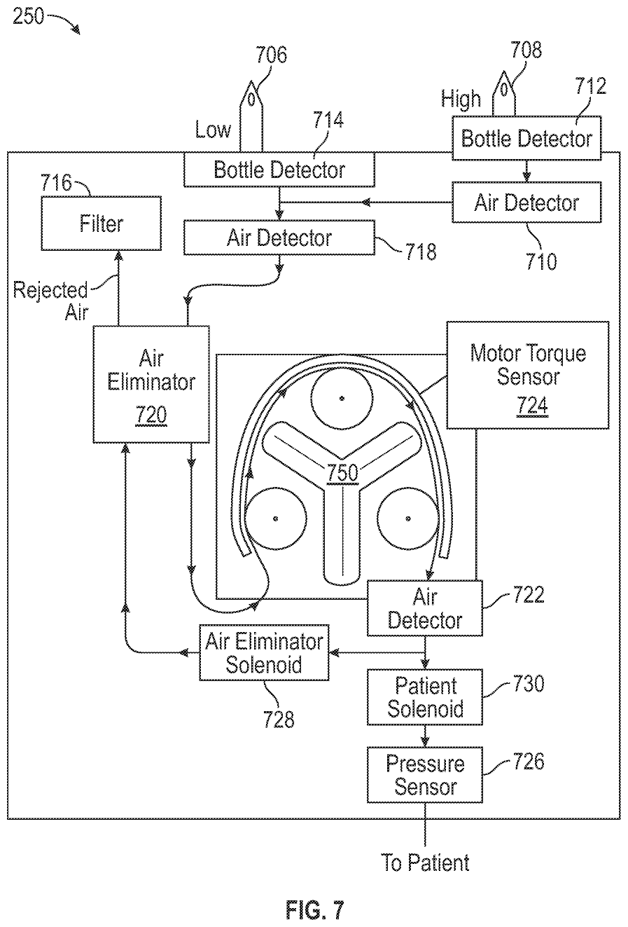

FIG. 7 illustrates a block diagram of an exemplary cartridge that may be used according to some embodiments. This example is a modification of the cartridge depicted in FIGS. 4A-4B.

In some embodiments, the cartridge 250 includes a low spike 706 port and a high spike 708 port. The cartridge includes air detectors 710 and 718, bottle detectors 712 and 714, a filter 716, an air eliminator 720, a motor torque sensor 724, and a patient pressure sensor 726. The air detectors 710 and 718 detect air in the fluid pathway. The bottle detectors 712 and 714 detect the presence of a medication bottle attached to the spikes 706 and 708. A filter 716 filters discharge of air (with possible fluid) from the cartridge 250.

In this embodiment, the cartridge 250 includes an air eliminator solenoid 728 and a patient solenoid 730. The device 100 controls the solenoids to open and close fluid pathways to and from the respective solenoids 728 and 730.

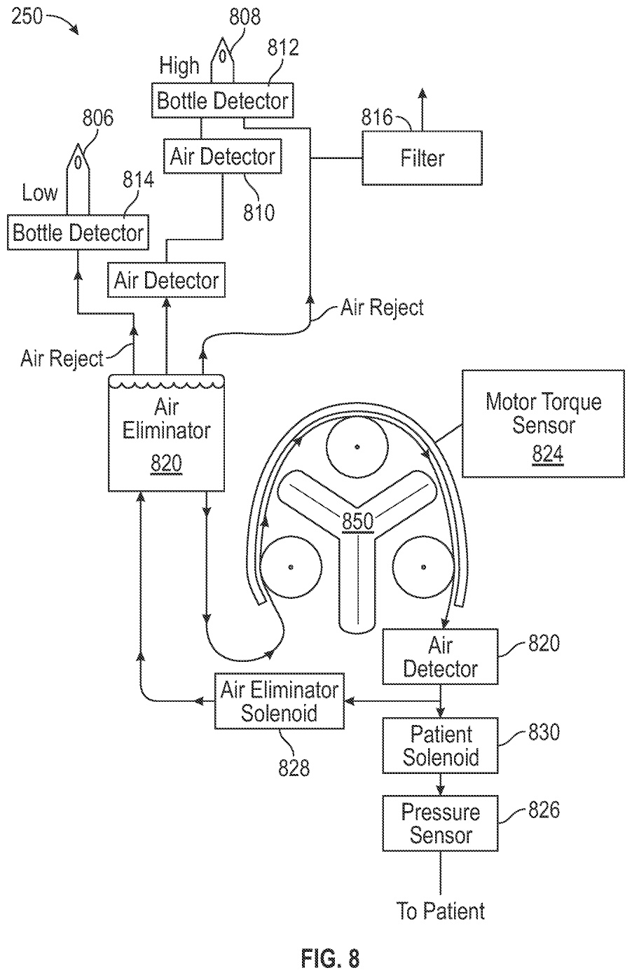

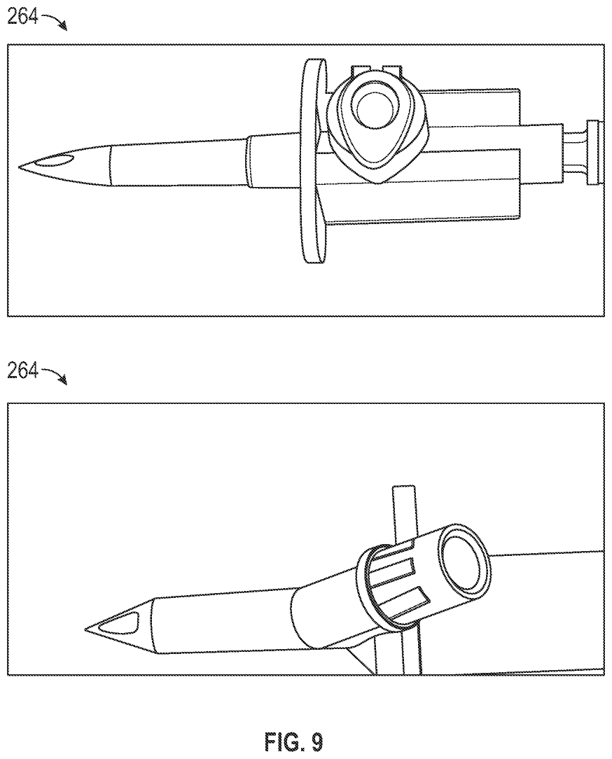

FIG. 8 illustrates a block diagram of an exemplary cartridge that may be used according to some embodiments. This example is a modification of the cartridge depicted in FIGS. 4A-4B.

In some embodiments, the cartridge 250 includes a low spike port 806 and a high spike port 808. The cartridge includes air detectors 810 and 818, bottle detectors 812 and 814, a filter 716, an air eliminator 820, a motor torque sensor 824, and a patient pressure sensor 826. The air detectors 810 and 818 detect air in the fluid pathway. The bottle detectors 812 and 814 detect the presence of a bottle attached to the spikes 806 and 808. A filter 816 filters discharge of air (with possible fluid) from the cartridge 250.

In this embodiment, the cartridge 250 includes an air eliminator solenoid 828 and a patient solenoid 830. The device 100 controls the solenoids to open and close fluid pathways to and from the respective solenoids 828 and 830.

In this embodiment, the air eliminator 820 provides an air elimination pathway to the low spike 806 port, the high spike port 808, and the filter 816.

FIG. 9 illustrates a perspective view of exemplary spikes 264 that may be used according to some embodiments. The spikes 264 may be fluidly connected to the device 100 and serve as the low spike and high spike ports as described herein.

### MODES OF OPERATION

In some embodiments, the device 100 performs a cartridge initialization operation. For example, the device 100 operates the pump to receive medication or fluid from an attached bottle. The device 100 automatically causes the fluid to fill the fluid pathways of the cartridge tubing thereby eliminating air or gas in the tubing. In some embodiments, the device senses when air has been evacuated from the tubing and then ceases the cartridge initialization operation. Once the air has been evacuated from the tubing, the device 100 may indicate that the device is ready for patient attachment and medication dispensing.

In some embodiments, the device 100 continuously monitors various parameters for anomalies. The device 100 determines anomalous situations and provides an audible alert and/or warning about the situations via a device 100 speaker. These audible alerts may further be transmitted to the EHR for alert and/or documentation, a remote monitoring location, and/or a smart device and/or headphones if attached. Moreover, the device 100 may provide audible descriptions of certain operation values of the device 100 (such as dosage information, flow rate, remaining volume of an attached bottle). The device 100 may provide audible feedback for pause, resume and dose change, and other audible alerts or warnings. The audible alert may be in the form of tones, beeps, and/or the alert may be in the form of haptic feedback, such as from a smart device. Moreover, the device 100 may generate audio signals describing the situation and/or operational values in a spoken language (such as the English language), especially useful if transmitted to the headphone(s) of an anesthesia professional.

In some embodiments, the device 100 operates in an auto loading mode. As dose is increased, the device 100 automatically gives additional, loading (catch-up) boluses and/or briefly speeds delivery of medication to achieve a new steady-state blood level of medication and therefore the patient's brain sedation level more rapidly and predictably. In the opposite situation, as dose is decreased, timed pauses and/or decreases in infusion speeds will help lower steady-state patient blood levels of medication and therefore sedation levels more rapidly and predictably. For example, without the auto-loading mode active, it may take significant time for dosing changes executed by the device 100 to be realized at steady-state bloodstream levels and therefore patient brain sedation levels. In some embodiments, the device 100 provides a graphical control, button or input icon that is displayed via the display device 120 to toggle the auto loading mode on/off.

In some embodiments, the device 100 operates in a Context Sensitive Half Time (CSHT) mode. In this mode, the device will periodically decrease the dose rate to match patient metabolism changes. For example, the initial doses of medication can be cleared by metabolism quite rapidly; however, the patient clears less and less as more medication is given and metabolic systems are taxed and/or exhausted. This CSHT mode may be accomplished by either: 1) lengthening the dosing interval time with no change to dose delivered in that interval; or 2) decreasing the dose delivered during the interval with no change to the interval time (both 1 & 2 will decrease dose per unit time). This decrease in the current dose rate of the medication during infusion serves to maintain steady blood levels of medication and therefore sedation effect as metabolism changes (decreases). For example, CSHT mode may decrease dose rate by 1% every 30 minutes of the infusion or utilize a more complex/exponential mathematical methodology. This decrease may be based on patient nomograms or may be otherwise experimentally derived. If CSHT mode is OFF, then the device does not adjust dosing downward, however, the device estimates TTFR (time to full recovery) (i.e., time until wake up) metric. The TTFR metric increases as total sedation time is longer and with higher dosing rates. For example, the TTFR metric may increase by 1% for every 30 minutes of sedation at normal levels or utilize a more complex/exponential mathematical methodology. This TTFR may be based on patient nomograms or may be experimentally derived. This TTFR may be displayed on the screen or otherwise communicated to the user to assist in efficient patient adjustments or wake up, assisting the anesthesia professional. In some embodiments, the device 100 provides a graphical control, button or input icon that is displayed via the display device 120 to toggle the CSHT mode on/off.

In some embodiments, the CSHT mode will adjust dose slightly downward over long spans of time (hours). This will match a medication profile of the dispensed medication to maintain stable and appropriate blood levels over time, compensating for changes (decreases) in metabolism by the patient over time. This will help to maintain a stable level of patient sedation over longer sedations. If CSHT is off, TTFR metric will increase and be displayed.

In some embodiments, the device 100 displays one or more graphical indications via the display device 120 of certain operational situations. For example, the device may display an “attention” graphic (such as a triangle with an exclamation point, with accompanying up/down arrows) for a situation where a “dose is high” (i.e., above a programmed maximum, with up arrows) and/or where a “dose is low” (i.e., below a programmed minimum, with down arrows). In some embodiments, these “attention” graphics will include an audible and/or visual flash alarm. Additionally, an information graphic (such as a circle with “i”) may be displayed to indicate “touch here to further explain” functions (such as when the device is first used by a new user or not in an expert mode of operation).

In some embodiments, the device 100 provides a graphical control, button or input icon that is displayed via the display device 120. A user of the device 100 may select this graphical control to switch operation of the device 100 to/from a new user mode (regular operational mode) and an expert (experienced user) operational mode. The expert operational mode would not include the information graphic (such as a circle with “i”) as discussed above. In some embodiments, user identification through biometric or other means would be necessary to allow unlocking of expert mode (for example anesthesia professionals may be allowed to unlock and activate expert mode but nursing may not). In some embodiments, additional settings/overrides may be allowed in expert mode. In some embodiments, expert mode detailed behavior and/or settings may only be accessible by a supervising physician, identified by biometric and/or other means.

In some embodiments, the device 100 operates in “One Button Case Start” mode. In this mode of operation, the device 100 will give a starting (induction of anesthesia) bolus of medication (for example, for the induction of anesthesia, which may or may not be calculated on a patient weight basis) and after cancellation (by user override) or delivery of this full amount of medication, then the device 100 would immediately switch to continuous maintenance infusion (dosing rate) mode. In the continuous maintenance infusion mode, the device 100 periodically delivers and verifies a dose of a medication according to preset dosage parameters. In this way, the anesthesia professional may start a sedation case with a single button push.

In some embodiments, the device 100 operates in a patient-controlled anesthesia (PCA) mode. In this PCA mode of operation, the device 100 uses a sensor (wireless or wired) attached to the patient's hand. With patient movement and/or button press of triggering device coupled to the device 100, the device may deliver a supplemental dose of medication (usually narcotic pain medication) within preset dosage parameters (for example, if such a dose is “due” and/or “allowed”). This mode may be useful for patients on a ventilator being sedated in intensive care unit (ICU) environments. The device 100 may use a keyed box, keypad locking code (or other security measure) outside of an operating room environment to prevent unauthorized changes/adjustments to dosage of the medication by unauthorized users.

In some embodiments, the device operates in a “Quick Bolus” mode. In this mode of operation, when the user requests by interface “one touch” with display device 120 and/or smart device and/or EHR interface, the device 100 delivers a brief additional amount of medication to quickly deepen anesthesia and then immediately resume current infusion speed.

In some embodiments, and in some modes of operation, the device 100 displays a total dose and current dosing of medication via the display device 120. The device 100 continuously monitors and tracks the amount of medication that is administered to a patient via the device 100. While the total dose and current dosing is displayed on the display device 120, this information may also be communicated to a separate device or system (EHR and/or smart device via Bluetooth or Wi-Fi) for remote monitoring of the dosing of the medication.

In some embodiments, the device 100 tracks the total amount of medication dispensed via the cartridge 250. For example, the device may determine a number of rotations of a rotatable gear that actuates a pump. The device 100 may equate a rotation of the gear to a particular amount of fluid dispensed via the pump. By tracking the total number of rotations of the rotatable gear, the device may determine a total amount of fluid dispensed by the cartridge.

In some modes of operation, the device 100 controls the pump 254 in a reverse flow operation. For example, the pump 254 can briefly be run in reverse at the patient connection (prior to patient disconnect) to remove any remaining medication from the connection hub (for example at patient wake up, or if air is detected by an air detector and/or visualized inside the tubing by the anesthesia professional). This removed medication may be disposed through a separate, dedicated disposal pathway.

As will be understood by one skilled in the art, other medications can be delivered with the disclosed infusion pump. Using cartridge identification technology, limited infusions (e.g., 100 mL of antibiotic) can be infused easily and automatically documented to the EHR with minimal effort, assisting the anesthesia professional.

As described previously, a removable cartridge 250 may be inserted to a cartridge receiver 160. The device 100 identifies the cartridge and controls the operation of a medication dispensing controller (e.g., a pump 254) to prepare the cartridge for use and dispense medication according to dosing parameters. The dosing parameters set the amount of the medication (total or rate) that will be dispensed via the medication dispensing controller 254.

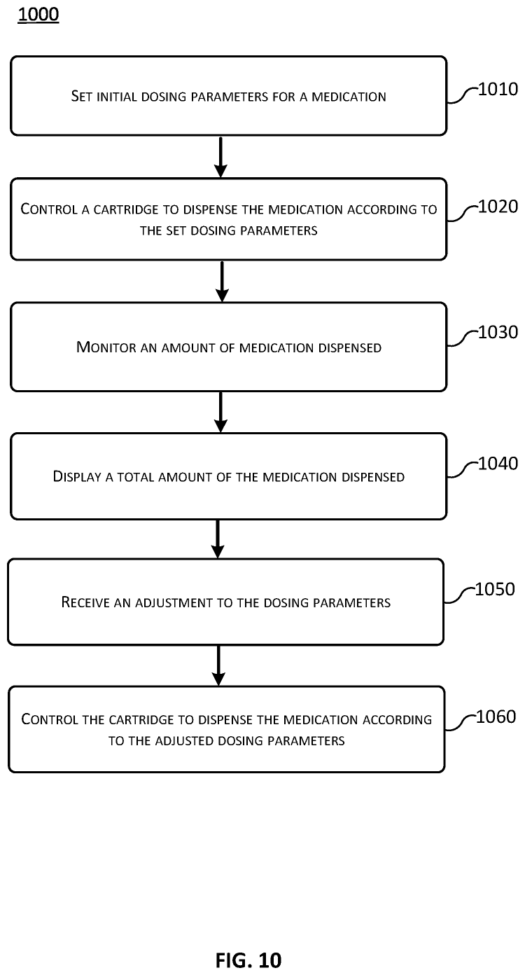

FIG. 10 illustrates an exemplary method of operation of an intuitive total intravenous anesthesia infusion system. The system operates to dispense medication via a removable cartridge.

In step 1010, the system sets initial dosing parameter to dispense a medication by the system.

In step 1020, the system controls the removable cartridge to dispense the medication according to the set dosing parameters. The cartridge comprises a medication dispensing controller and one or more fluid pathways.

In step 1030, the system continuously monitors the amount of medication having been dispensed via the cartridge. The system also continuously monitors for anomalies in the operation of the device and the delivery of a medication.

In step 1040, the system displays, via a display device, a total amount of the medication having been dispensed and/or a current dosing rate.

In step 1050, the system receives an adjustment to the initial dosing parameters.

In step 1060, the system recalculates, then adjusts the dosing rate and controls the removable cartridge to dispense medication according to the adjusted dosing parameters.

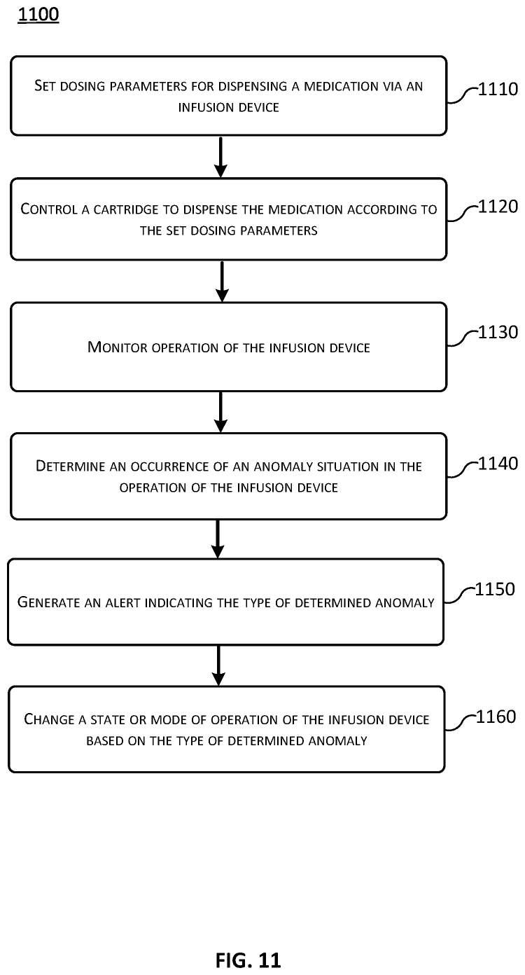

FIG. 11 illustrates an exemplary method of operation of an intuitive total intravenous anesthesia infusion system. While dispensing a medication via an infusion device 100, the device detects anomalous situations relating to the operation of the device 100.

In step 1110, the system sets initial dosing parameter to dispense a medication by via an infusion device 100.

In step 1120, the system controls a removable cartridge to dispense the medication according to the set dosing parameters. The cartridge comprises a medication dispensing controller and one or more fluid pathways.

In the 1130, the device 100 continuously monitors the operation of the infusion device and evaluates the operation of the device for one or more anomalous operational situations.

In step 1140, the device 100 determines an anomaly situation in the operation of the device 100. For example, the device 100 may monitor for: air detected in a fluid pathway of the cartridge 250; the amount of fluid received from an attached medication bottle to the cartridge and whether a bottle is empty or near empty; a low battery voltage of the device; and/or detecting pressure indicating an occlusion situation of a dispensed medication.

In some embodiments, the device 100 may determine a torque value of a gear rotating a pump in the cartridge 250. If the torque value meets or exceeds a predetermined threshold torque value, then the device may determine the occurrence of an anomaly in the operation of the pump. This situation could mean that the cartridge pump is inoperable or that the cartridge 250 may not be seated properly in the cartridge receiver or may assist in the detection of a patient IV infiltration and/or occlusion situation.

In some embodiments, the device 100 may determine that a medication bottle is low or empty and/or that bottle has been removed from a spike. For example, the device may include a bottle detector that determines whether a bottle has been placed on to a spike. In some embodiments, the bottle detector is of a push button type where the bottle actuates the push button when the bottle is seated onto the spike. When the button is pushed inwards, the device determines that a bottle is connected to the port. If the bottle is removed, the button springs upward, and the device 100 determines that a bottle has been removed and/or a bottle is not connected to the port. The device 100 may determine an anomaly situation where a bottle is removed from the device 100.

In some embodiments, the device 100 may determine an empty bottle situation. For example, air detectors of the cartridge may detect the occurrence of air or gas in the fluid pathway. The device 100 may determine an anomaly situation where a bottle is detected as empty.

In step 1150, the device 100 generates an alert indicating the type of determined anomaly. Based on the determined anomaly, the device provides an audible indication describing the anomaly situation.

In step 1160, the device 100 changes a state or mode of operation of the infusion device based on the type of determined anomaly. For certain detected anomalies, the device 100 may only provide an audible warning and continue to operate normally. For example, if a low battery voltage is detected, the device may provide an audible alert, but continue to dispense the medication according to the dispensing parameters. For other detected anomalies, such as an occlusion situation, the device may provide an audible alert and automatically pause the operation of the medication dispensing controller.

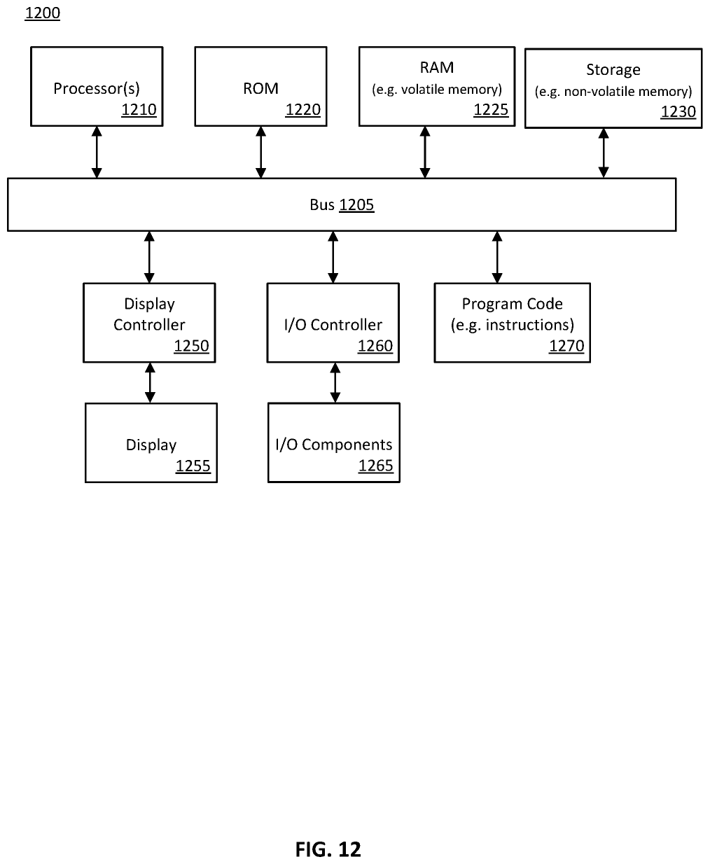

FIG. 12 shows a block diagram of an example of a computing system that may be used in conjunction with one or more embodiments of the disclosure. For example, computing system 1200 (or system, or server, or computing device, or device) may represent any of the devices or systems described herein that perform any of the processes, operations, or methods of the disclosure. Note that while the computing system 100 illustrates various components, it is not intended to represent any particular architecture or manner of interconnecting the components as such details are not germane to the present disclosure. It will also be appreciated that other types of systems that have fewer or more components than shown may also be used with the present disclosure.

As shown, the computing system 1200 may include a bus 1205 which may be coupled to a processor 1210, ROM (Read Only Memory) 1220, RAM (or volatile memory) 1225, and storage (or non-volatile memory) 1230. The processor(s) 1210 may retrieve stored instructions from one or more of the memories 1220, 1225, and 1230 and execute the instructions to perform processes, operations, or methods described herein. These memories represent examples of a non-transitory computer-readable medium (or machine-readable medium, a computer program product, etc.) containing instructions (or program code) which when executed by a processor (or system, device, etc.), cause the processor to perform operations, processes, or methods described herein.

As referred to herein, for example, with reference to the claims, a processor may include one or more processors 1210. The performance of operations may be distributed among the one or more processors 1210, whether residing only within a single machine or deployed across a number of machines. The RAM 1225 may be implemented as, for example, dynamic RAM (DRAM), or other types of memory that require power continually in order to refresh or maintain the data in the memory. Storage 1230 may include, for example, magnetic, semiconductor, tape, optical, removable, non-removable, and other types of storage that maintain data even after power is removed from the system. It should be appreciated that storage 1230 may be remote from the system (e.g., accessible via a network).

A display controller 1250 may be coupled to the bus 1205 in order to receive display data to be displayed on a display device 1255, which can display any one of the user interface features or embodiments described herein and may be a local or a remote display device. The computing system 1200 may also include one or more input/output (I/O) components 1265 including mice, keyboards, touch screen, network interfaces, printers, speakers, and other devices. Typically, the input/output components 1265 are coupled to the system through an input/output controller 1260.

Program code 1270 may represent any of the instructions, applications, software, libraries, toolkits, modules, components, engines, units, functions, logic, etc. as described herein (e.g., system 100, user interface module 202, dosing adjustment module 204, cartridge interface module 20, bar code reading module 210, EHR interface module 212, anomaly detection module 214, and radio frequency module 216.) Program code 1270 may reside, completely or at least partially, within the memories described herein (e.g., non-transitory computer-readable media), or within a processor during execution thereof by the computing system. Program code 1270 may include both machine code, such as produced by a compiler, and files containing higher-level or intermediate code that may be executed by a computing system or other data processing apparatus (or machine) using an interpreter. In addition, program code 1270 can be implemented as software, firmware, or functional circuitry within the computing system, or as combinations thereof. Program code 670 may also be downloaded, in whole or in part, through the use of a software development kit or toolkit that enables the creation and implementation of the described embodiments.

In one implementation, the program code 1270 includes instructions to implement functionality corresponding to the components of a device to perform the disclosure herein. While the machine-readable storage medium is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

In general, the terms “engine” and “module”, as used herein, refer to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on one or more computer readable media, such as compact discs, digital video discs, flash drives, or any other tangible media. Such software code may be stored, partially or fully, on a memory device of the executing computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware. Generally, the modules described herein refer to logical modules that may be combined with other modules or divided into sub-modules despite their physical organization or storage

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

It will be appreciated that the present disclosure may include any one and up to all of the following examples.

Example 1. An intravenous medication infusion system, comprising: a body comprising one or more onboard processors; a display device operably connected to the one or more onboard processors; an input control; a cartridge receiver; and a removable cartridge comprising a medication dispensing controller; wherein the one or more onboard processors are configured to perform the operations comprising: setting dosing parameters for the dosing of a medication; and controlling the medication dispensing controller to dispense an amount of the medication via the removable cartridge.

Example 2. The system of Example 1, wherein the removable cartridge comprises a reservoir that holds the medication, and wherein the medication dispensing controller is a pump that is fluidly connected to the reservoir.

Example 3. The system of any one of Examples 1-2, wherein the cartridge contains a reusable portion and a disposable portion. The disposable portion may be swapped and reloaded to save on environmental impact and save cost. The reusable portion may be manufactured for indefinite use/reuse.

Example 4. The system of any one of Examples 1-3, wherein the onboard processor is further configured to perform the operations comprising: after receiving the removable cartridge into the cartridge receiver, priming a tubing connected to the cartridge according to the tubing size/shape/volume and medication dispensing configuration data.

Example 5. The system of any one of Examples 1-4, wherein the onboard processor is further configured to perform the operations comprising: displaying, via the display device, current dosing parameters for the medication; receiving an input via the input control; based on the received input, adjusting the dosing parameters to different dosing parameters; and dispensing by the medication dispensing controller, an amount of the medication according to the different dosing parameters.

Example 6. The system of any one of Examples 1-5, wherein the onboard processor is further configured to perform the operations comprising: displaying, via the display device, a patient weight value; receiving an input via the input control; based on the received input, adjusting the patient weight value; based on the adjusted patient weight value, adjusting the dosing parameters of the medication; and dispensing by the medication dispensing controller, an amount of the medication according to the adjusted dosing parameters.

Example 7. The system of any one of Examples 1-6, wherein the onboard processor is further configured to perform the operations comprising: establishing a connection with a separate EHR system; and transmitting to the separate EHR system the data related to the dosing and delivery rate of the medication.

Example 8. The system of any one of Examples 1-7, wherein the onboard processor is further configured to: monitoring an amount of medication that has been dispensed via the medication dispensing controller; periodically determining a total amount of the medication dispensed; and displaying, via the display device, the total amount of the medication dispensed.

Example 9. The system of any one of Examples 1-8, wherein the input control comprises a rotatable knob that changes the dosing parameters when the knob is pushed inwards toward the body and/or when the knob is rotated.

Example 10. The system of any one of Examples 1-9, wherein the display device comprises a touch input screen configured to display a selection of one or more commands generated by the onboard processors and the display device is configured to receive an input via the touch input screen for the selection of a command, and wherein the one or more onboard processors adjust the dosing parameters of the medication according to the selected command.

Example 11. The system of any one of Examples 1-10, wherein the one or more onboard processors are further configured to perform the operations of: determining an anomalous situation with the dispensing of the medication; and providing an audible alert or warning regarding the determined anomalous situation; wherein the determined anomalous situation is any one or more of the following: air detected in a fluid pathway of the cartridge; whether a bottle attached to the cartridge is nearly empty or empty; a low battery voltage of the device; and/or an occlusion situation of a dispensed medication.

Example 12. A method of operating an intravenous infusion device, the method comprising: providing the intravenous infusing device, the device comprising: a body comprising one or more onboard processors; a display device operably connected to the one or more onboard processors; an input control; a cartridge receiver; and a removable cartridge comprising a medication dispensing controller; setting dosing parameters for the dosing of a medication via the device; and controlling the medication dispensing controller to dispense an amount of the medication via the removable cartridge.

Example 13. The method of Example 12, wherein the removable cartridge comprises a reservoir that holds the medication, and wherein the medication dispensing control is a pump that is fluidly connected to the reservoir.

Example 14. The method of any one of Examples 11-13, further comprising: after receiving the removable cartridge into the cartridge receiver, priming a tubing connected to the cartridge.

Example 15. The method of any one of Examples 11-14, further comprising: displaying, via the display device, current dosing parameters for the medication; receiving an input via the input control; based on the received input, adjusting the dosing parameters to different dosing parameters; and dispensing by the medication dispensing controller, an amount of the medication according to the different dosing parameters.

Example 16. The method of any one of Examples 11-15, further comprising: displaying, via the display device, a patient weight value; receiving an input via the input control; based on the received input, adjusting the patient weight value; based on the adjusted patient weight value, adjusting the dosing parameters of the medication; and dispensing by the medication dispensing controller, an amount of the medication according to the adjusted dosing parameters.

Example 17. The method of any one of Examples 11-16, further comprising: establishing a connection with a separate EHR system; and transmitting to the separate EHR system the data related to the dosing and delivery rate of the medication.

Example 18. The method of any one of Examples 11-17, further comprising: monitoring an amount of medication that has been dispensed via the medication dispensing controller; periodically determining a total amount of the medication dispensed; and displaying, via the display device, the total amount of the medication dispensed.

Example 19. The method of any one of Examples 11-18, wherein the input control comprises a rotatable knob that changes the dosing parameters when the knob is pushed inwards toward the body and/or when the knob is rotated.

Example 20. The method of any one of Examples 11-19, wherein the display device comprises a touch input screen configured to display a selection of one or more commands generated by the onboard processors and the display device is configured to receive an input via the touch input screen for the selection of a command, and wherein the one or more onboard processors adjust the dosing parameters of the medication according to the selected command.

Example 21. The method of any one of Examples 11-20, further comprising: determining an anomalous situation with the dispensing of the medication; and providing an audible alert or warning regarding the determined anomalous situation; wherein the determined anomalous situation is any one or more of the following: air detected in a fluid pathway of the cartridge; whether a bottle attached to the cartridge is nearly empty or empty; a low battery voltage of the device; and/or an occlusion situation of a dispensed medication.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. An intravenous medication infusion system, comprising:
a body comprising one or more onboard processors;
a display device operably connected to the one or more onboard processors;
an input control;
a cartridge receiver; and
a removable cartridge comprising a medication dispensing controller;
wherein the one or more onboard processors are configured to perform the operations comprising:
setting dosing parameters for the dosing of a medication; and
controlling the medication dispensing controller to dispense an amount of the medication via the removable cartridge.

2. The system of claim 1, wherein the removable cartridge comprises a reservoir that holds the medication, and wherein the medication dispensing controller is a pump that is fluidly connected to the reservoir.

3. The system of claim 2, wherein the onboard processor is further configured to perform the operations comprising:
after receiving the removable cartridge into the cartridge receiver, priming a tubing, connected to and/or incorporated in the cartridge, with an amount of medication.

4. The system of claim 1, wherein the onboard processor is further configured to perform the operations comprising:
displaying, via the display device, current dosing parameters for the medication;
receiving an input via the input control;
based on the received input, adjusting the dosing parameters to different dosing parameters; and
dispensing by the medication dispensing controller, an amount of the medication according to the different dosing parameters.

5. The system of claim 1, wherein the onboard processor is further configured to perform the operations comprising:
displaying, via the display device, a patient weight value;
receiving an input via the input control;
based on the received input, adjusting the patient weight value;
based on the adjusted patient weight value, adjusting the dosing parameters of the medication; and
dispensing by the medication dispensing controller, an amount of the medication according to the adjusted dosing parameters.

6. The system of claim 1, wherein the onboard processor is further configured to perform the operations comprising:
establishing a connection with a separate EHR system; and
transmitting to the separate EHR system the data related to the dosing and delivery rate of the medication; and
receiving from the separate EHR system parameters, including patient weight.

7. The system of claim 1, wherein the onboard processor is further configured to:
monitoring an amount of medication that has been dispensed via the medication dispensing controller;
periodically determining a total amount of the medication dispensed; and
displaying, via the display device, the total amount of the medication dispensed.

8. The system of claim 1, wherein the input control comprises a rotatable knob that changes the dosing parameters when the knob is pushed inwards toward the body and/or when the knob is rotated.

9. The system of claim 1, wherein the display device comprises a touch input screen configured to display a selection of one or more commands generated by the onboard processors and the display device is configured to receive an input via the touch input screen for the selection of a command, and wherein the one or more onboard processors adjust the dosing parameters of the medication according to the selected command.

10. The system of claim 1, wherein the one or more onboard processors are further configured to perform the operations of:
determining an anomalous situation with the dispensing of the medication; and
providing an audible alert or warning regarding the determined anomalous situation;
wherein the determined anomalous situation is any one or more of the following: air detected in a fluid pathway of the cartridge; whether a bottle attached to the cartridge is nearly empty or empty; a low battery voltage of the device; and/or an occlusion situation of a dispensed medication.

11. A method of operating an intravenous infusion device, the method comprising:
providing the intravenous infusing device, the device comprising:
a body comprising one or more onboard processors;
a display device operably connected to the one or more onboard processors;
an input control;
a cartridge receiver; and
a removable cartridge comprising a medication dispensing controller;
setting dosing parameters for the dosing of a medication via the device; and
controlling the medication dispensing controller to dispense an amount of the medication via the removable cartridge.

12. The method of claim 11, wherein the removable cartridge comprises a reservoir that holds the medication, and wherein the medication dispensing control is a pump that is fluidly connected to the reservoir.

13. The method of claim 11, further comprising:
after receiving the removable cartridge into the cartridge receiver, priming a tubing connected to the cartridge according to the medication dispensing configuration data.

14. The method of claim 11, further comprising:
displaying, via the display device, current dosing parameters for the medication;
receiving an input via the input control;
based on the received input, adjusting the dosing parameters to different dosing parameters; and
dispensing by the medication dispensing controller, an amount of the medication according to the different dosing parameters.

15. The method of claim 11, further comprising:
displaying, via the display device, a patient weight value;
receiving an input via the input control;
based on the received input, adjusting the patient weight value;
based on the adjusted patient weight value, adjusting the dosing parameters of the medication; and
dispensing by the medication dispensing controller, an amount of the medication according to the adjusted dosing parameters.

16. The method of claim 11, further comprising:
establishing a connection with a separate EHR system; and
transmitting to the separate EHR system the data related to the dosing and delivery rate of the medication; and
receiving from the separate EHR system parameters, such as patient weight.

17. The method of claim 11, further comprising:
monitoring an amount of medication that has been dispensed via the medication dispensing controller;
periodically determining a total amount of the medication dispensed; and
displaying, via the display device, the total amount of the medication dispensed.

18. The method of claim 11, wherein the input control comprises a rotatable knob that changes the dosing parameters when the knob is pushed inwards toward the body and/or when the knob is rotated.

19. The method of claim 11, wherein the display device comprises a touch input screen configured to display a selection of one or more commands generated by the onboard processors and the display device is configured to receive an input via the touch input screen for the selection of a command, and wherein the one or more onboard processors adjust the dosing parameters of the medication according to the selected command.

20. The method of claim 11, further comprising:
determining an anomalous situation with the dispensing of the medication; and
providing an audible alert or warning regarding the determined anomalous situation;
wherein the determined anomalous situation is any one or more of the following: air detected in a fluid pathway of the cartridge; whether a bottle attached to the cartridge is nearly empty or empty; a low battery voltage of the device; and/or an occlusion situation of a dispensed medication.
